(19) United States
(12) Patent Application Publication
(10) Pub. No.: US 2010/0174066 A1
(43) Pub. Date: Jul. 8, 2010

(54) PROCESS FOR PREPARING CRYSTALLINE FORM I OF OLANZAPINE

(76) Inventors: Venkataraman Sundaram, Hyderabad (IN); Sharat Narsapur Pandurang, Hyderabad (IN); Vishal Parmar Dayaram, Maharashtra (IN); Siva Kumar Reddy Bommareddy, Hyderabad (IN); Hitendra Chaudhary Sitaram, Hyderabad (IN)

Correspondence Address: DR. REDDY''S LABORATORIES, INC., 200 SOMERSET CORPORATE BLVD, SEVENTH FLOOR, BRIDGEWATER, NJ, 08807-2862, US

(21) Appl. No.: 11/909,275

(22) PCT Filed: Mar. 20, 2006
(86) PCT No.: PCT/US06/09911

§ 371 Date: Sep. 21, 2007

Related U.S. Application Data
(60) Provisional application No. 60/677,115, filed on May 3, 2005.

(30) Foreign Application Priority Data
Mar. 21, 2005 (IN) 291/CHE/2005

(51) Int. Cl. C07D 487/04

(52) U.S. Cl. 540/557

(57) ABSTRACT

A process for preparing olanzapine Form I comprises: a) cooling a concentrated solution of olanzapine; b) isolating wet crystals of olanzapine Form I; and c) drying wet crystals and recovering olanzapine Form 1. Drying can be conducted by stepwise increases in the drying temperatures, with extended holding times at each temperature condition.

### INTRODUCTION TO THE INVENTION

The present invention relates to a process for the preparation of substantially pure Form I of olanzapine.

Olanzapine is chemically named 2-methyl-4-(4-methyl-1-piperazinyl)-10H-thieno[2,3-b][1,5]benzodiazepine and is structurally represented by Formula I.

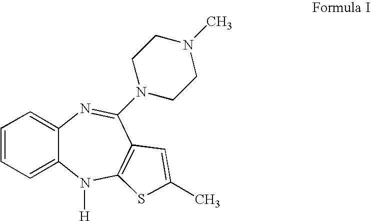

Olanzapine is an antagonist of dopamine at D-1 and D-2 receptors. In addition, it has antimuscarinic and anti-cholinergic properties and antagonistic activity at 5-hydroxytryptophan-2 (5-HT2) receptor sites. It also has antagonistic activity at noradrenergic alpha-receptors. It is a potential neuroleptic with relaxant properties, has anxiolytic or anti-emetic properties, and is useful in treating psychotic conditions such as schizophrenia, schizophreniform disease, and acute mania. At lower doses the compound is indicated for use in the treatment of mild anxiety states. Olanzapine can be used both in its free base and acid addition salt forms. It is commercially available in the market under the brand name ZYPREXA™ as tablets containing the equivalent of 2.5, 5, 7.5, 10, 15, and 20 mg of olanzapine.

U.S. Pat. No. 5,229,382 describes the preparation of olanzapine or an acid addition salt thereof by reacting N-methyl piperazine with 4-amino-2-methyl-10H-thieno[2,3-b][1,5]benzodiazepine hydrochloride. The anhydrous form of olanzapine obtained by the process of this patent is referred to herein as “Form I.” Form I has been reported to be metastable and to change its color on standing.

Polymorphic forms and hydrates/solvates of olanzapine have been disclosed in various patents and publications. For example, U.S. Pat. No. 5,736,541 and EP Patent No. 733,635 disclose olanzapine Form II anhydrate.

U.S. Application Publication No. 2004/0067936 A1 discloses a laboratory-scale process for the preparation of Form I of olanzapine comprising recrystallization in dichloromethane and drying the wet compound in an oven at 60 to 70° C. Form I of olanzapine prepared according to the process described in this patent application is contaminated with olanzapine Form II, when manufactured on an industrial scale.

U.S. Application Publication No. 2003/0125322 A1 discloses a process for the preparation of stable crystalline Form I of olanzapine having a stable color at ambient conditions of storage, free from the solvent of crystallization i.e. dichloromethane. The process disclosed uses rotary driers like Rota-cone and horizontal rotary vacuum dryers for drying the final solid.

International Application Publication No. WO 2005/080401 A1 claims a process for the preparation of olanzapine Form I comprising drying the final product under vacuum at a temperature of 60 to 70° C. for a period ranging from 36 to 60 hours or 45 to 50 hours without breaking the vacuum.

International Application Publication No. WO 2003/097650 teaches that the use of any solvent such as acetonitrile, tetrahydrofuran, acetone, toluene, or ethyl acetate for crystallization of crude olanzapine always results in crystalline Form II. The only solvent enabling the formation of polymorphic Form I of olanzapine by crystallization of crude olanzapine or of its crystalline Form II is methylene chloride. The application discloses the conversion of Form II of olanzapine to Form I by recrystallization in methylene chloride. The product obtained is dried in an air oven at 60 to 70° C.

U.S. Application Publication No. 2004/0048854 describes a process for the preparation of olanzapine Form I free from polymorphic Form II as a contaminant by applying a different pH in its solution state with different organic solvents. The patent discloses Form I which contains less than 2 percent of Form II.

Methylene chloride is a Class 2 solvent and the upper limit of residual methylene chloride in the final product according to the ICH guidelines (International Conference for Harmonization) is 600 ppm. Thus, olanzapine Form I for use in pharmaceutical formulations has to be dried to an extent where the concentration of residual methylene chloride is less than 600 ppm, requiring extended drying as in the above-mentioned patents.

All of the above mentioned patents for the preparation of olanzapine Form I utilize air tray drying or the vacuum tray drying for drying of the final product. These drying techniques require the product to be exposed to elevated temperatures for prolonged periods of time to reduce the residual methylene chloride levels to within the limits given in the ICH guidelines.

Regulatory authorities worldwide require that olanzapine to be used in pharmaceutical compositions should be substantially free from contamination by other polymorphs and residual solvents. There is thus a great felt need for a reproducible method for the preparation of Form I of olanzapine substantially free from the crystalline Form II. There is also a need for a reproducible process for the preparation of such a pure Form I of olanzapine that is readily scalable and operable on an industrial scale.

The present invention provides a process for the preparation of substantially pure olanzapine Form I free of Form II of olanzapine and other volatile impurities. The process is easily scalable and provides reproducible results.

### SUMMARY OF THE INVENTION

The present invention provides a reproducible process for the preparation of substantially pure olanzapine Form I having a low concentration of the crystalline Form II.

One aspect of the invention provides a process for the preparation of substantially pure olanzapine Form I having a low concentration of the crystalline Form II.

An embodiment of a process for the preparation of substantially pure olanzapine Form I comprises the steps of:
a) providing a solution of olanzapine;
b) concentrating the solution to a minimum volume and cooling the remaining concentrate to lower temperatures;
c) isolating the solid to get wet crystals of olanzapine Form I;
d) drying the wet crystals obtained to get substantially pure olanzapine Form 1.

An aspect of the invention involves the drying of the wet crystals of olanzapine Form I using a fluidized bed dryer.

Another aspect of the invention comprises drying wet crystals of olanzapine Form I by exposing wet crystals to temperatures about 35° C., then increasing temperature in increments of about 10° C. to a final temperature about 75° C.

A further aspect of the invention comprises drying by exposing wet crystals of olanzapine Form I to temperatures about 35 to 40° C. for about 1 to 4 hours, 45 to 50° C. for about 1 to 4 hours, 50 to 65° C. for about 1 to 4 hours, 60 to 65° C. for about 1 to 4 hours, and 70 to 75° C. for about 1 to 4 hours.

Yet another aspect of the invention provides olanzapine Form I substantially free of Form II.

Still another aspect of the invention provides olanzapine Form I substantially free of residual solvents.

An embodiment of the invention includes a process for making olanzapine Form I, substantially free from Form II, the process comprising drying wet crystals of olanzapine Form I at temperatures about 35° C., then increasing temperatures in a stepwise manner with predetermined holding times at each temperature condition.

### BRIEF DESCRIPTION OF THE DRAWINGS

FIG. 1 is an X-ray powder diffraction (“XRPD”) pattern of crystalline olanzapine Form I.

FIG. 2 is an XRPD pattern of crystalline olanzapine Form II.

FIG. 3 is an XRPD pattern of Form I of olanzapine that contains 0.5 percent w/w of Form II.

FIG. 4 is a quantification curve using XRPD for substantially pure olanzapine Form I.

### DETAILED DESCRIPTION OF THE INVENTION

In one aspect, the invention provides a reproducible process for the preparation of substantially pure olanzapine Form I, having a low concentration of the crystalline Form II.

The term “reproducible process” means a process that produces a product of a specified quality on a consistent basis.

A embodiment of a process for the preparation of substantially pure olanzapine Form I comprises the steps of:
a) providing a solution of olanzapine;
b) concentrating the solution to a minimum volume and cooling the concentrate to lower temperatures;
c) isolating the solid to get wet crystals of olanzapine Form I; and
d) drying the wet crystals obtained to get substantially pure olanzapine Form I.

Step a) involves providing a solution of olanzapine.

The solution of olanzapine can be obtained by dissolving olanzapine in methylene chloride. Any form of olanzapine is acceptable for forming the solution, such as any crystalline or amorphous form of olanzapine including any salts, solvates with alcohols such as methanol, ethanol, or propanol, and hydrates including the monohydrate, dihydrate, etc. The solution can also be obtained directly from a reaction in which olanzapine is formed.

The solution can be prepared at a temperature ranging from about 25° C. to 50° C. Depending on the quantity of solvent taken, it may dissolve at 25 to 35° C., or the solution may need to be heated to elevated temperatures of about 40° C. to 50° C.

The solution may optionally be treated with carbon or with sodium sulfate for clarification.

The solution can optionally be filtered by passing through paper, glass fiber, or other membrane material or a clarifying agent such as celite. Depending upon the equipment used and the concentration and temperature of the solution, the filtration apparatus may need to be preheated to avoid premature crystallization.

The concentration of the solution can be about 1 g/ml to about 20 g/ml of the solvent, or it may range form 1 g/ml to 5 g/ml.

Step b) involves concentration of the olanzapine solution.

Concentration may be carried out suitably using evaporation, atmospheric distillation or distillation under vacuum.

Distillation of the solvent may be conducted under a vacuum of about 100 mm Hg to about 600 mm Hg at lower temperatures of about 5° C. to about 30° C. Any temperature and vacuum conditions can be used as long as concentration occurs without an increase in the impurity levels.

Concentration of the solution can be carried out to an extent where the precipitation of the Form I of olanzapine begins from the solution, converting the solution into a slurry. Generally, concentration will be terminated when the ratio of solvent to olanzapine becomes about 1:1 weight/volume.

After the completion of the concentration, the reaction mass may be maintained further at temperatures lower than the concentration temperatures such as for example below about 40° C. to about 45° C., for a period of time as required for a more complete isolation of the product. The exact cooling temperature and time required for complete crystallization can be readily determined by a person skilled in the art and will also depend on parameters such as concentration and temperature of the solution or slurry.

Step c) involves isolation of the wet crystals.

The solid isolation can be conducted by conventional techniques such as filtering, decanting, centrifuging and the like, or by filtering under an inert atmosphere using gases such as for example nitrogen and the like.

Isolation of the olanzapine wet crystals can be conducted at lower temperatures of 5 to 10° C. because of the instability of solution of olanzapine in methylene chloride at higher temperatures. Higher or lower temperatures are acceptable as long as there is no further generation of impurities.

The isolated solid may optionally be washed with chilled methylene chloride, at a temperature of below about 20 to 25° C. The choice of temperature will of course depend on the solubility of the crystals at that temperature. A temperature at which the solubility is the lowest is preferred to optimize yields.

Step d) involves drying of the wet cake using a suitable technique.

The wet cake is further dried to obtain olanzapine Form I substantially free of residual methylene chloride solvents and moisture and substantially free of crystalline Form II.

Suitable drying techniques which can be used for drying olanzapine wet crystals include without limitation fluidized bed drying, flash drying, spin flash drying, microwave drying, and the like. Other drying techniques which could help in drying the product in a short period of time, are easily scaleable, and also produce reproducible results.

In one embodiment of the invention drying of the wet cake of olanzapine Form I is accomplished with the use of a fluidized bed dryer (“FBD”).

FBD is a versatile drying method particularly suitable for products like olanzapine Form I and helps in obtaining a product which is free of contamination with other forms, especially Form II, and also having a reduced residual solvent content.

In brief, the feed of wet materials is dried by intimate contact with hot air which is passed through the material which is in a fluidized state. A vibrating mechanism can be attached to give a forward motion cum agitation to the product at a controlled rate. It is a very versatile process equipment for uniform and efficient drying of products with minimum handling and is more hygienic.

The process achieves rapid heat transfer making it very efficient, yet gentle on the product.

A fluidized bed is a bed of solid particles with a stream of air or gas passing upward through the particles at a rate great enough to set them in motion. As the air travels through the particle bed, it imparts unique properties to the bed. For example, the bed behaves like a liquid. It is possible to propagate wave motion, which creates the potential for improved mixing. Thus, the fluid bed can be used to dry the wet product, deagglomerate particles, improve flow properties, or produce coated particles for controlled release or taste masking.

Some fluid bed dryers used in the present invention utilize programmable temperature controllers.

Air is fed at slightly positive atmospheric pressures.

The wet crystals of olanzapine can be exposed to a humid atmosphere having a relative humidity typically less than about 70%, or less than about 40%. The time of exposure varies, and depends on the amount of material.

The fluidization is carried out at temperatures which are gradually increased starting from about 35° C. in a programmed stepwise manner. The product is maintained at each temperature range for different time intervals.

In an embodiment, the temperature for drying is varied by about 10° C. starting from about 35° C. to about 75° C. At each temperature condition, the product is maintained for about 1 to 4 hours. This gradual increase of temperature avoids the continuous exposure of the product to higher temperatures. The whole drying process can take from about 4 hours to about 10 hours, or longer.

In another embodiment, a programmed stepwise rise in temperature with appropriate holding times chosen at each temperature may be for example 35 to 40° C. for 1 to 4 hours, 45 to 50° C. for 1 to 4 hours, 50 to 65° C. for 1 to 4 hours, 60 to 65° C. for 1 to 4 hours, 70 to 75° C. for 1 to 4 hours.

The foregoing times for different temperature conditions are only representative of those that can be used to make a product having low levels of residual solvent. Particularly at the lower drying temperatures, the times can be prolonged beyond about 4 hours as desired. A determination of appropriate times can be easily made by a person skilled in the art, using simple experimentation, to produce a product having desired characteristics.

The programmed temperature variation provides the advantage of drying of the compound at lower temperatures. The compound need not be exposed to a constant high temperature for a long time. The material is exposed to higher temperatures for a short period of time. This drying technique also lowers the methylene chloride content to the required limits as per the ICH guidelines. The drying process takes a shorter span and is easily scalable for industrial purposes and the results obtained are reproducible.

The dried product can be optionally milled to get the required particle size. Milling or micronization can be performed intermediary to drying, or after the completion of drying of the product. The milling operation reduces the size of particles and increases surface area of particles by colliding particles with each other at high speeds.

Drying is more efficient when the particle size of the material is smaller and the surface area is higher, hence milling can be performed intermediary to the drying operation.

A fluid energy mill, or micronizer, is one of the techniques to produce particles of small size in a narrow size distribution. These mills use the kinetic energy of collision between particles suspended in a rapidly moving fluid stream to cleave the particles. For milling of olanzapine Form I, an air jet mill can be used, which is a fluid energy mill. The suspended particles are injected under pressure into a recirculating particle stream. Smaller particles are carried aloft inside the mill and swept into a vent connected to a particle size classifier such as a cyclone.

Other methods of particle size reduction, which can be used, are conventional ball, roller, or hammer mills.

Olanzapine Form I is characterized by its XRPD pattern. The XRPD data reported herein were obtained using Cu Kα radiation, having the wavelength 1.541 Å, and generated on a Bruker Axe, D8 Advance Powder X-ray Diffractometer.

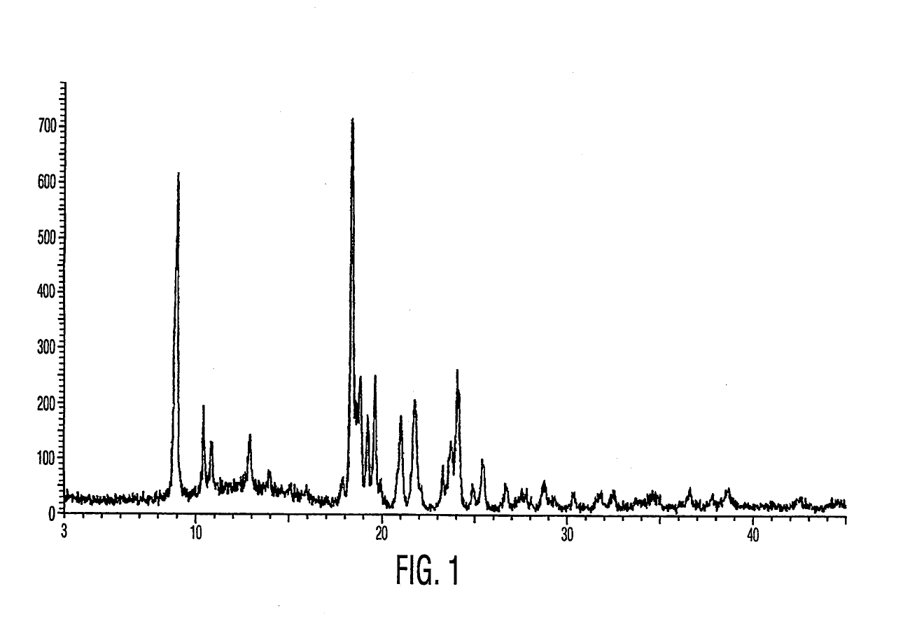

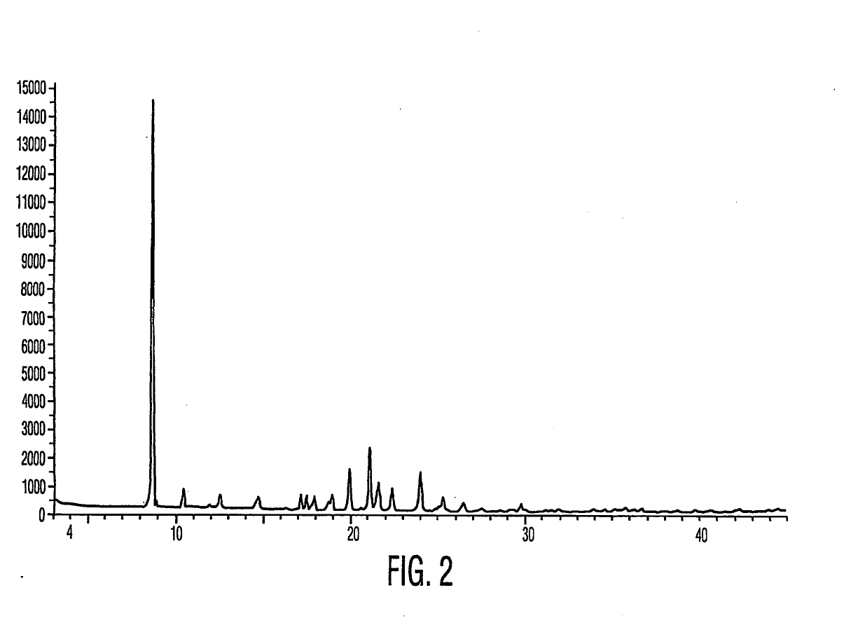

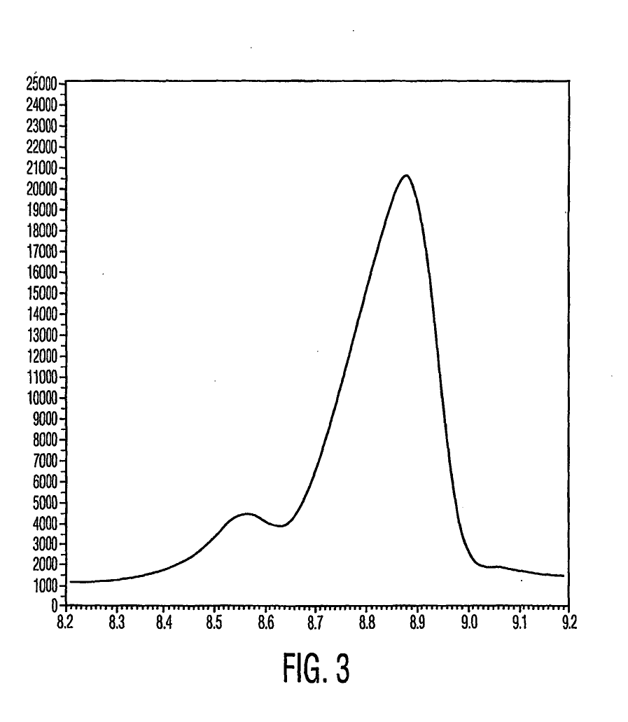

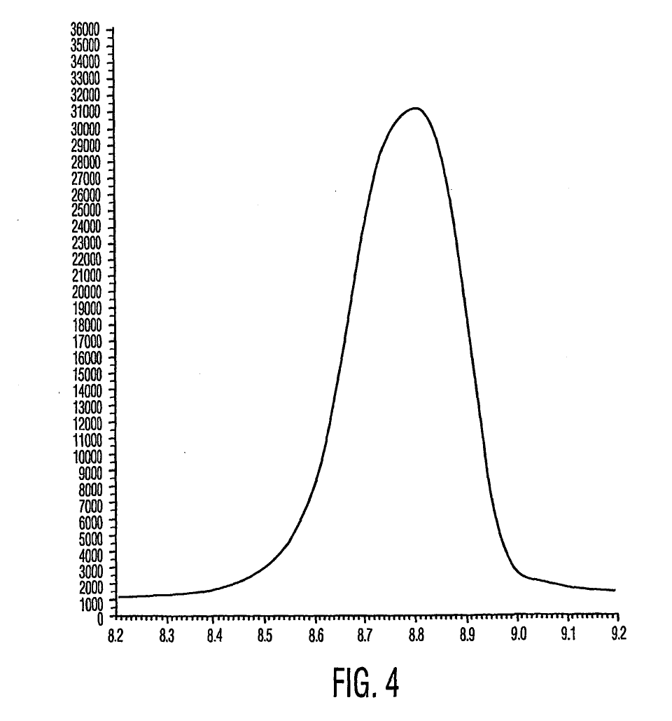

Olanzapine Form I and Form II are characterized by their XRPD patterns substantially in accordance with FIGS. 1 and 2, respectively. Form II of olanzapine contaminating Form I of olanzapine can be analyzed with XRPD. The method used herein can detect and quantify as little as 0.5 weight percent of Form II in Form I. The quantification curve for olanzapine Form II in Form I of this invention is given in FIG. 3, and the quantification curve for pure Form I is FIG. 4. In each of these drawing figures, the vertical axis is intensity and the horizontal axis is the 2θ angle, in degrees.

Yet another aspect of the invention provides olanzapine Form I substantially free of residual solvents.

Olanzapine Form I obtained using this process contains less than 500, or less than 400, ppm of methylene chloride, and less than 500 ppm of any other volatile impurities. The solvent content can be analyzed by gas chromatography.

Still another aspect of the invention provides substantially pure stable olanzapine Form I having a low concentration of olanzapine Form II.

As used herein “substantially pure” refers to Form I associated with less than about 5 percent by weight Form II, or less than about 2 percent Form II, or less than about 0.5 percent Form II.

The present invention and the advantages provided thereby will be more fully understood with reference to the following non-limiting examples, which describe certain aspects and embodiments of the invention but are not to be construed as limiting the invention.

### Example 1

### Preparation of Wet Crystals of Olanzapine

2.5 kg of olanzapine monohydrate was taken into a reactor and 45 liters of methylene chloride was added. The suspension was heated to 43° C. to obtain a clear solution. The resultant solution was filtered through a perlite bed in a pressure Nutch filter. The filtered bed was washed with methylene chloride. The clear filtrate was distilled under vacuum at a temperature of 10° C. to a volume of 12.5 liters. The reaction mass was then cooled to 3° C. and stirred at the same temperature for 60 minutes. The isolated solid was filtered and washed with 2.5 liters of chilled methylene chloride to get 1.63 kg of the title compound.

### Comparative Example

### Experiment to Demonstrate the Heat Sensitivity of Olanzapine

Olanzapine Form I wet crystals obtained by following the process similar to that given in Example 1 were taken and dried at different temperatures in an air tray drier for 8 hours to check the impact on the crystalline purity of Form I obtained. The results are tabulated below in Table 1:

TABLE 1

| Drying Temperature | |
|---|---|
| (° C.) | Form II content (%) |
| 60 | Less than 0.5 |
| 75-80 | Less than 0.5 |
| 100 | Less than 0.5 |
| 125 | More than 0.5 |
| 150 | More than 0.5 |
| 180 | More than 0.5 |

*Quantification is done by XRPD and the limit of quantification (LOQ) is about 0.5% by weight.

### Example 2

### Experiment to Compare the Drying Techniques for Olanzapine Form I

The wet olanzapine Form I obtained according to the procedure given in Example 1 was subjected to different drying techniques both at laboratory scale and pilot plant scale. The results are tabulated in Table 2.

TABLE 2

| | Methylene chloride | | Form II content | |
|---|---|---|---|---|
| | content (ppm) | | after drying | |
| Type of | | Pilot plant | | Pilot plant |
| dryer | Lab scale | scale | Lab scale | scale |
| Air tray dryer | 300-600 | 300-600 | Not detected | Traces of |
| (10 hours) | | | | Form II |
| | | | | present |
| Vacuum | Less than | More than 600 | Not detected | Form II |
| oven | 600 | | | observed in |
| (10 hours) | | | | some cases |
| Fluidized bed | Less than | Much less than | Not detected | Not detected |
| drying (3 | 600 | 600 | | |
| hours) | | | | |
| Bucci | Less than | Less than 600 | Not detected | Good |
| Rotavapor | 600 | | | amount |
| | | | | of Form II |
| | | | | observed |
| Microwave | Below 600 | — | Not detected | — |
| oven drying | | | | |

It is evident from the results that techniques like air tray drying and vacuum tray drying although suitable at lab scale, are not suitable for drying at plant scale. Fluidized bed drying and microwave drying provide the desired results.

### Example 3

### Preparation of Substantially Pure Form I of Olanzapine Using Fluidized Bed Drying

4.0 kg of the wet cake of olanzapine obtained according to Example 1 was dried in a fluid bed dryer with an air flow rate of 200 cubic feet per minute (CFM) with stepwise rise in temperature from 45° C. to 70° C. and maintaining the wet material at each temperature range for 1 to 4 hours as per Table 3. The dried material was unloaded to yield 3.2 kg of olanzapine Form I substantially free from Form II.

Residual dichloromethane content: 167 ppm.
Form II Content: Less than 0.5%.
Moisture content: 0.2%
FIG. 1 is an XRPD pattern for olanzapine Form I obtained above.
FIG. 4 gives the quantification curve for olanzapine Form II in Form I. The quantification curve shows that the Form II content in the sample is less than 0.5%.
Table 3 shows programmed temperature variation followed for Example 3.

TABLE 3

| Duration | Temperature |
|---|---|
| (Minutes) | (° C.) |
| 30 | 41 |
| 45 | 43 |
| 30 | 50 |
| 15 | 55 |
| 30 | 63 |
| 20 | 65 |
| 30 | 68 |
| 30 | 69 |

### Example 4

### Preparation of Substantially Pure Form I of Olanzapine Using Microwave Drying

10 g of the wet compound obtained in Example 1 was subjected to microwave drying, using a standard kitchen microwave oven, at a power output of 900 Watts for 10 minutes. The dried material was unloaded and characterized using XRPD. Form I of olanzapine substantially free from Form II was obtained. Residual dichloromethane content: 300-400 ppm.

### Example 5

### Detection of Olanzapine Form II in Form I by X-Ray Diffraction

### Standard Preparation:

497.5 mg of olanzapine Form I and 2.5 mg of olanzapine Form II (weight ratio of olanzapine Form Ito Form II of 99.5:0.5) were weighed and mixed with 50.0 mg of lithium fluoride. The mixture was mixed and ground three times using a mortar and pestle made of agate to get a homogeneous fine powder. A maximum quantity of the finely ground sample was packed in a sample holder and this was used as the standard for powder X-ray diffraction analysis.

### Test Sample Preparation:

500 mg of olanzapine Form I test sample was weighed and mixed with 50 mg of lithium fluoride. The mixture was mixed and ground three times using a mortar and pestle made of agate to get a homogeneous fine powder. A maximum quantity of the finely ground sample was packed in a sample holder and this was used as the test sample for the analysis.

Experimental conditions for performing the XRPD analysis for quantification were as follows:

Instrument used: Powder X-ray diffractometer.

Make, Model: Bruker AXS, D8 Advance.

Goniometer: Theta/theta vertical.

Measuring circle: 435 nm.

Radiation: Cu K alpha-1 (Wavelength=1.5406 Å).

Tube: 2.2 kw Copper long fine focus.

Detector: Scintillation counter.

Voltage, current: 40 kV, 50 mA.

Scan type: Locked couple.

Scan mode: Step scan.

Divergence Slit: 1.0 degree.

Antiscattering Slit: 1.0 degree.

Detector slit: 0.2 nm.

Synchronous rotation: On.

Scan Range: 8.2° to 9.2° 2θ.

Step size 0.004 degrees.

Time/step: 10.0 seconds.

The standard and the test sample were analyzed and the intensity at 8.60° (2θ) in the diffractogram of the test sample was compared with respect to that of the 0.5% standard. If the test sample intensity at 8.60° (2θ) is more than that of the 0.5% standard, then the presence of Form II in Form I is more than 0.5%. If the test sample intensity at 8.60° (2θ) is less than that of the 0.5% standard, then the presence of Form II in Form I is less than 0.5%.

1. A process for preparing olanzapine Form I, comprising:
a) cooling a concentrated solution of olanzapine;
b) isolating wet crystals of olanzapine Form I; and
c) drying the wet crystals and recovering olanzapine Form I.

2. The process of claim 1, wherein the solution in step a) comprises methylene chloride.

3. The process of claim 1, wherein the concentrated solution of olanzapine in step a) is prepared by removing solvent from the solution until crystals form.

4. The process of claim 3, wherein the concentrated solution has a ratio of solvent to olanzapine of about 1:1.

5. The process of claim 1, wherein drying in step c) comprises exposing the wet crystals to initial temperatures of about 35° C., then increasing the temperature in a stepwise manner with predetermined holding times at each temperature condition.

6. The process of claim 1, wherein drying in step c) comprises exposing the wet crystals to initial temperatures of about 35° C., then increasing the temperature in increments of about 10° C. to a final temperature of about 75° C.

7. The process of claim 6, wherein the initial temperature and each temperature increment is independently maintained for at least about one hour.

8. The process of claim 1, wherein drying in step c) comprises exposing the wet crystals to temperatures of about 35 to 40° C. for about 1 to 4 hours, 45 to 50° C. for about 1 to 4 hours, 50 to 65° C. for about 1 to 4 hours, 60 to 65° C. for about 1 to 4 hours, and 70 to 75° C. for about 1 to 4 hours.

9. The process of claim 1, wherein drying in step c) is conducted using a fluid bed dryer.

10. The process of claim 1, wherein the recovered olanzapine Form I has a residual solvent content less than about 500 ppm.

11. The process of claim 1, wherein the recovered olanzapine Form I contains less than about 5 weight percent of olanzapine Form II.

12. A process for preparing olanzapine Form I, comprising:
a) cooling a concentrated solution of olanzapine in methylene chloride;
b) isolating wet crystals of olanzapine Form I; and
c) drying the wet crystals, by exposing the wet crystals to temperatures of about 35° C., then increasing the temperature in a stepwise manner with predetermined holding times at each temperature condition, and recovering olanzapine Form I.

13. The process of claim 12, wherein the concentrated solution of olanzapine in step a) is prepared by removing solvent from the solution until a ratio of olanzapine to methylene chloride is about 1:1.

14. The process of claim 12, wherein drying in step c) comprises exposing the wet crystals to temperatures of about 35° C., then increasing the temperature in increments of about 10° C. to a final temperature of about 75° C.

15. The process of claim 12, wherein drying in step c) comprises exposing the wet crystals to temperatures of about 35 to 40° C. for about 1 to 4 hours, 45 to 50° C. for about 1 to 4 hours, 50 to 65° C. for about 1 to 4 hours, 60 to 65° C. for about 1 to 4 hours, and 70 to 75° C. for about 1 to 4 hours.

16. The process of claim 12, wherein drying in step c) is conducted using a fluid bed dryer.

17. The process of claim 12, wherein the recovered olanzapine Form I has a residual methylene chloride content less than about 500 ppm.

18. The process of claim 12, wherein the recovered olanzapine Form I contains less than about 5 weight percent of olanzapine Form II.

19. The process of claim 12, wherein the recovered olanzapine Form I contains less than about 2 weight percent of olanzapine Form II.

20. The process of claim 12, wherein the recovered olanzapine Form I contains less than about 0.5 weight percent of olanzapine Form II.

21. A process for making olanzapine Form I, substantially free from Form II, the process comprising drying wet crystals of olanzapine Form I at initial temperatures of about 35° C., then increasing the temperature in a stepwise manner with predetermined holding times at each temperature condition.

22. The process of claim 21, wherein drying comprises exposing the wet crystals to initial temperatures of about 35° C., then increasing the temperature in increments of about 10° C. to a final temperature of about 75° C.

23. The process of claim 21, wherein the initial temperature and each temperature increment is independently maintained for at least about one hour.

24. The process of claim 21, wherein drying comprises exposing the wet crystals to temperatures of about 35 to 40° C. for about 1 to 4 hours, 45 to 50° C. for about 1 to 4 hours, 50 to 65° C. for about 1 to 4 hours, 60 to 65° C. for about 1 to 4 hours, and 70 to 75° C. for about 1 to 4 hours.

25. The process of claim 21, wherein drying is conducted using a fluid bed dryer.
